(19) United States
(12) Patent Application Publication
(10) Pub. No.: US 2025/0389727 A1
(43) Pub. Date: Dec. 25, 2025

(54) Dissociation Method and System of Deprotonated Peptides with Fragile Moieties

(71) Applicant: DH Technologies Development Pte. Ltd., Singapore (SG)

(72) Inventors: Takashi BABA, Richmond Hill (CA); Haichuan LIU, Redwood City, CA (US); Yuzhuo ZHANG, Redwood City, CA (US)

(21) Appl. No.: 18/881,363

(22) PCT Filed: Jun. 29, 2023
(86) PCT No.: PCT/IB2023/056799

§ 371 Date: Jan. 6, 2025

Related U.S. Application Data
(60) Provisional application No. 63/358,752, filed on Jul. 6, 2022.

(51) Int. Cl. G01N 33/68; H01J 49/00; H01J 49/04

(52) U.S. Cl. CPC G01N 33/6848; H01J 49/0054; H01J 49/0481

(57) ABSTRACT

A method for mass spectrometric analysis of a peptide having at least one fragile moiety includes using electrospray ionization to generate a negatively charged ion of said peptide, trapping and cooling the negatively charged peptide ion in a radiofrequency (RF) ion trap containing a cooling buffer gas, and exposing said cooled, trapped peptide ion to an electron beam so as to cause negative electron activated dissociation (negative EAD) of the negatively charged peptide ion to generate a plurality of fragment ions.

### TECHNICAL FIELD

The following relates to a mass spectrometer and more particularly to a mass spectrometer utilizing electron activation dissociation (EAD) including electron detachment dissociation (EDD) and negative electron induced dissociation (EID) applied to negatively charged analyte ions.

### BACKGROUND

Mass spectrometry (MS) is an analytical technique for determining the elemental composition of a substance. Specifically, MS measures a mass-to-charge ratio (m/z) of ions generated from a test substance. MS can be used to identify unknown compounds, to determine isotopic composition of elements in a molecule, to determine the structure of a particular compound by observing its fragmentation, and to quantify the amount of a particular compound in a sample. Mass spectrometers detect ions and as such, a test sample must be converted to an ionic form (i.e., precursor ions or analyte ions) during mass analysis. Generally, a mass spectrometer includes at least an ion source, a mass analyzer, and an ion detector. The ion source converts a test sample into gaseous ions, the analyzer separates the gaseous ions based on their m/z ratios, and the detector detects the separated ions. In many mass spectrometers for biomolecule analysis, dissociation (or fragmentation) devices are installed in the mass spectrometers.

A mass spectrometer can employ EAD to cause the fragmentation of analytes into smaller fragment ions. These fragment ions can then be mass analyzed and quantified based on their m/z ratios. EDD works on negatively multiply charged analyte ions, such as peptides, oligonucleotides, DNA, RNA, etc. Negative EID works on negatively charged singly charged analyte ions, such as acidic peptides, fatty acids and acidic complex lipids. As used herein, negative EAD includes both EDD and negative EID.

### SUMMARY

Aspects of the present disclosure address the above-referenced problems and/or others.

In one aspect, a method for mass spectrometry is disclosed, which includes using an electrospray ionization source to ionize a plurality molecules of a peptide having a fragile moiety, e.g., generate a plurality of negatively charged peptide ions (e.g., deprotonated peptides) with a fragile moiety. By way of example, the fragile moiety can be coupled via one or more chemical bonds to the peptide. In some cases, the fragile moiety can be the result of a post translational modification (PTM) of the original peptide. The method further includes trapping the peptide ions in an RF ion trap that includes a buffer gas. In the ion trap, the peptide ions can undergo cooling collisions with the molecules of the buffer gas, which result in the cooling of the peptide ions. The method also includes exposing the cooled peptide ions to an electron beam that causes the peptide ions to fragment via negative EAD, while ensuring that the majority of (and preferably all of) the fragile moieties remain chemically connected to the backbones of one or more fragment ions generated via negative EAD. In some embodiments, the fragmentation of the cooled peptide ions can be achieved with minimal, and preferably no dissociation of the fragile moieties from the backbone of the peptide. In some embodiments, negative EAD includes EDD or negative EID. In some embodiments, the electron beam has a kinetic energy in a range of about 20 eV to about 50 eV.

In some embodiments, the RF ion trap can be a linear RF ion trap. In some embodiments, the RF trap can be a branched RF ion trap. An RF ion trap can include a plurality of rods arranged in a multipole configuration. The multipole configuration can include any of a quadrupole, a hexapole, an octupole, and a dodecapole configuration. The buffer gas can include any of nitrogen, helium, neon, argon, xenon, and any other suitable molecule. In some embodiments, the method further includes acquiring a mass spectrum of the fragment ions.

In another aspect, a mass spectrometer includes an ion source configured to ionize a plurality of peptides that include a fragile moiety thereby generating a plurality of peptide ions that include a fragile moiety and a chamber. In some embodiments, the peptide ion is a negative ion. The chamber can include an ion trap that includes a buffer gas and is configured to trap the peptide ion and an electron source configured to generate a plurality of electrons in the form an electron beam and configured to introduce the electron beam into the ion trap. The molecules of the buffer gas cool the peptide ions within the ion trap, and the electron beam interacts with at least a portion of the peptide ions to cause negative EAD, thereby generating a plurality of fragment ions with a fragile moiety. In some embodiments, the negative EAD can be one of EDD or negative EID and the electron beam can have a kinetic energy in a range of about 20 eV to about 50 eV.

In some embodiments, the RF ion trap is a linear RF trap. In other embodiments, the RF ion trap is a branched RF ion trap. The RF ion trap can include a plurality of rods arranged in a multipole configuration. The multipole configuration can include any of a quadrupole, a hexapole, an octupole, and a dodecapole configuration. In some embodiments, the buffer gas includes any of nitrogen, helium, neon, argon, and xenon. In some embodiments, the mass spectrometer further includes a mass analyzer configured to receive the fragment ions and provide mass spectral data indicative thereof; and a mass analysis module configured to process the mass spectral data to generate a mass spectrum of the fragment ions.

A branched ion trap can include two sets of L-shaped electrodes that are axially separated from another, wherein each set of the L-shaped electrodes comprises four electrodes arranged in a quadrupole configuration, though in other embodiments other multipole configurations may also be employed. In some embodiments, an RF voltage source can be employed to apply RF voltages to each set of L-shaped electrodes to generate a quadrupolar electric RF field in the space between electrodes.

In yet another aspect, a chamber for use in a mass spectrometer includes an ion trap and an electron source that is external to the ion trap. The ion trap includes a plurality of negatively charged peptide ions that include a fragile moiety, and a buffer gas configured to reduce a vibrational state of the peptide ions. The electron source is configured to introduce an electron beam into the ion trap. The electron beam interacts with the peptide ions to generate a plurality of fragment ions with a fragile moiety.

### BRIEF DESCRIPTION OF THE DRAWINGS

Aspects of the present disclosure may take form in various components and arrangements of components, and in various steps and arrangements of steps. The drawings are only for illustration purpose of preferred embodiments of the present disclosure and are not to be considered as limiting.

Features of embodiments of the present disclosure will be more readily understood from the following detailed description take in conjunction with the accompanying drawings in which:

FIG. 1 is a flow chart of a method for mass spectrometric analysis of a peptide having at least one fragile moiety in accordance with an exemplary embodiment;

FIG. 2 schematically illustrates a mass spectrometer in accordance with an exemplary embodiment;

FIG. 3 is a MS/MS spectrum of sulfated peptide obtained via EDD in accordance with an exemplary embodiment;

FIG. 4 is a MS/MS spectrum of sulfated peptide obtained via EDD in accordance with an exemplary embodiment;

FIG. 5 is a MS/MS spectrum of sulfated peptide obtained via EID in accordance with an exemplary embodiment;

FIG. 6 is a MS/MS spectrum of sulfated peptide obtained via EID in accordance with an exemplary embodiment; and

FIG. 7 schematically illustrates a computer system in accordance with an exemplary embodiment.

### DETAILED DESCRIPTION

It will be appreciated that for clarity, the following discussion will explicate various aspects of embodiments of the present disclosure, while omitting certain specific details wherever convenient or appropriate to do so. For example, discussion of like or analogous features in alternative embodiments may be somewhat abbreviated. Well-known ideas or concepts may also for brevity not be discussed at any great detail. One of ordinary skill will recognize that some embodiments of the present disclosure may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the disclosure. The following detailed description of embodiments is not to be regarded as limiting the scope of the applicant's teachings in any manner.

As used herein, the terms “about” and, “substantially, and “substantially equal” refer to variations in a numerical quantity and/or a complete state or condition that can occur, for example, through measuring or handling procedures in the real world; through inadvertent error in these procedures; through differences in the manufacture, source, or purity of compositions or reagents; and the like. Typically, the terms “about” and “substantially” as used herein means 10% greater or lesser than the value or range of values stated or the complete condition or state. For instance, a concentration value of about 30% or substantially equal to 30% can mean a concentration between 27% and 33%. The terms also refer to variations that would be recognized by one skilled in the art as being equivalent so long as such variations do not encompass known values practiced by the prior art.

As used herein the term “and/or” includes any and all combinations of one or more of the associated listed items and may be abbreviated as “/”.

The term “a fragile moiety,” as used herein refers to a chemical group attached to a peptide where the likelihood that the fragile moiety is dissociated from the peptide's backbone when the peptide undergoes collision induced dissociation (CID) is greater than 50%, and in some cases the likelihood is 100%. In other words, in collection of such peptides exposed to CID, the majority of the fragile moieties are dissociated from the peptide's backbone when the peptide undergoes CID. Some examples of such fragile moieties include, without limitation, sulfate, glycan, and phosphoryl moieties, among others.

There is an interest in mass spectrometric analysis of a variety of peptides using tandem mass spectrometry in which precursor peptides are ionized and the peptide ions are fragmented, and the mass spectra of the fragments are acquired and analyzed. Some peptides precursors may include post-translational modification (PTM) moieties, e.g., moieties attached to the peptide after formation of the peptide. Many post-translational modification (PTM) moieties are fragile moieties (also referred to as a “labile moiety”). As used herein, a fragile moiety is a moiety that would be removed from a precursor peptide or a protein when subjected to collision induced dissociation (CID).

Since fragile moieties are removed from a peptide when subjected to CID, the moiety cannot be localized. Hence, traditional CID techniques cannot be used to sequence peptides with the position information of the fragile moieties.

Furthermore, an acidic peptide that undergoes electron capture dissociation (or positive EAD) may not efficiently produce positively charged (or protonated) precursor ions needed to analyze a precursor ion of interest. For example, fragmenting a sulfated peptide via EDD or EID is a slow and inefficient process that results in the loss of sulfation moiety.

The present disclosure generally relates to a method of performing mass spectrometry of peptides (herein also referred to as “precursors” or “precursor peptides”) having one or more fragile moieties via ionizing the peptides to generate negatively charged peptide ions, cooling the negatively charged peptide ions and causing negative EAD fragmentation of the cooled peptide ions to generate a plurality of fragment ions thereof with minimal, and preferably no dissociation, of the fragile moieties from the peptide's backbone. In other words, the fragment ions (or at least a majority thereof) retain the fragile moieties.

Further, the present teachings can be used for mass spectrometric analysis of acidic peptides via ionization of such precursor peptides in an ion source of a mass spectrometer operating in the negative mode (e.g., an electrospray ion source operating in the negative mode) to produce precursor ions in a deprotonated [M-nH]ⁿ⁻ form. It has been discovered that in some embodiments cooling of such negatively charged peptide ions can be an important step in ensuring that subsequent fragmentation of the ions via negative EAD will not result in dissociation of the fragile moieties from the peptide's backbone.

In particular, in absence of cooling of the peptide precursor ions, when EDD is employed to fragment the precursor ions, the combination of vibrational energy contained in the uncooled precursor ions and the energy supplied by the electron beam may induce preferential loss of one or more fragile moieties coupled to the peptide's backbone. Similarly, when employing EID, a precursor ion undergoing collisions with electrons of an electron beam is excited to an electronic excited state.

Accordingly, there is a need for methods for mass spectrometric analysis of peptides having fragile moieties, where such methods are capable of causing fragmentation of precursor ions associated with such peptides with minimal, and preferably no dissociation, of the fragile moieties from the peptide's backbone.

In one aspect, the present disclosure generally relates to methods for performing mass spectrometry and mass spectrometers that can be utilized to practice such methods in which a plurality of negatively charged peptide ions (e.g., deprotonated peptides) having at least one fragile moiety are trapped within an ion trap of a mass spectrometer. In some embodiments, the precursor peptide includes the fragile moiety as a result of a PTM, though the presence of the fragile moiety can be due to other processes as well. The ion trap can contain a buffer gas (also referred to as a “cooling buffer gas”), which can be supplied to the ion trap, e.g., via a reservoir that is in communication with the ion trap.

In the ion trap, the precursor peptide ions collide with molecules of the buffer gas such that the vibrational energy of the precursor ions is reduced (also referred to as “cooling”). Following sufficient cooling of the precursor peptide ions, an electron beam is introduced into the ion trap such that the electrons in the beam can interact with the cooled peptide ions to cause fragmentation of at least a portion of the peptide ions. Since the cooled peptide ions have a reduced vibrational and kinetic energy, the fragmentation occurs preferentially along the peptide backbone without no (or at most minimal) dissociation of the fragile of the fragile moiety.

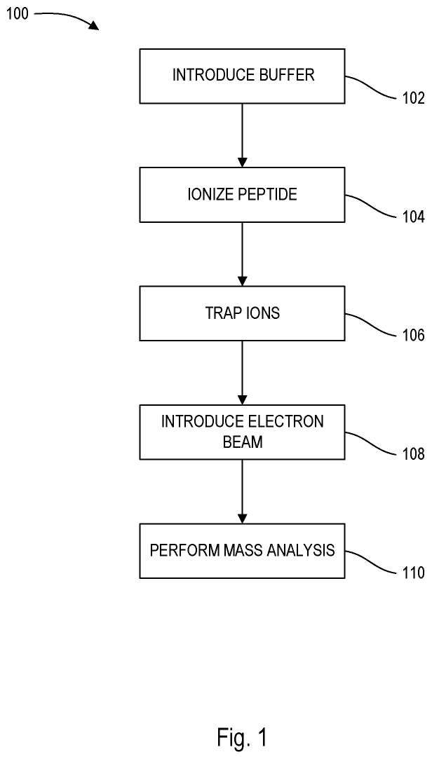

FIG. 1 is a flow chart depicting various steps in a method according to the present teachings for performing mass spectrometric analysis of peptides and in particular peptides having fragile moieties, e.g., in the form of pendant chemical groups attached to the peptide's backbone.

In this embodiment, the method includes using electrospray ionization to ionize a plurality of molecules of a peptide having at least one fragile moiety and cooling the peptide ions to reduce their vibrational and/or kinetic energy. By way of example, the precursor peptide ions can be introduced into an ion trap that contains molecules of a buffer gas, where the precursor ions can undergo cooling collisions with the buffer gas molecules to produce cooled precursor ions.

The cooled precursor ions can then be exposed to an electron beam so as to cause negative EAD of at least a portion of the cooled precursor ions, thereby generating a plurality of fragment ions with minimal, and in most embodiments, with no dissociation of the fragile moiety from the peptide's backbone. In some such embodiments, the precursor peptide ions are introduced into a branched RF ion trap, which contains a buffer gas, via an inlet thereof and are trapped within a reaction region of the ion trap. The precursor peptide ions are cooled via collisions with the buffer gas molecules. An electron beam is introduced into the ion trap, typically via a different inlet than the inlet utilized for the introduction of the precursor ions and along a direction that is generally perpendicular to the direction along which the precursor ions are introduced into the ion trap. The electrons in the electron beam interact with the precursor ion molecules to cause fragmentation of at least a portion thereof with minimal, and preferably no dissociation of the fragile moieties.

In some embodiments, the ion trap is maintained at a pressure in a range of about 0.1 milli to about 10 milli Torr. In some such embodiments, the precursor ions that are introduced into the ion trap have an energy in a range of about 0 eV to about 5 eV. In general, the energy of the precursor ions and the pressure of the buffer gas within the in trap are selected such that collisions of the precursor ions with the buffer gas molecules can preferentially cause collisional cooling of the precursor ions rather than their fragmentation via CID.

With continued reference to the flow chart of FIG. 1, the resultant fragment ions can be mass analyzed to generate a mass spectrum thereof and the mass spectrum of the fragment ions can be utilized to derive structural information regarding the precursor ions. In the case of EDD of deprotonated peptides, a-type and x-type fragment ions are produced. In the case of negative EID of deprotonated peptides a type and multiple types of C terminal fragment ions are produced. These fragmentation patterns are used to reconstruct the alignment of the amino acid residues in the tested peptides.

Referring now to FIG. 1, a method 100 for mass spectrometric analysis of a peptide having at least one fragile moiety is shown in accordance with an exemplary embodiment.

At 102, a buffer gas is introduced into an ion trap of the mass spectrometer as will be discussed in further detail herein.

At 104, an electrospray ion source generates peptide ions having at least one fragile moiety as will be discussed herein.

At 106, the peptide ions are trapped within the ion trap that contains the buffer gas to cool the peptide ions as will be discussed in further detail herein.

At 108, the cooled, trapped peptide ions are exposed to an electron beam which causes the peptide ions to fragment as will be discussed in further detail herein.

At 110, a mass analyzer receives the fragment ions and generates mass spectral data corresponding to m/z ratios of the ions as previously discussed herein. Furthermore, at 210 an analysis module receives the mass spectral data generated by the mass analyzer and processes the data to generate a mass spectrum of the fragment ions and correlates the mass spectrum of the fragment ions with peptide ions from which the fragment ions were generated as will be discussed in further detail herein.

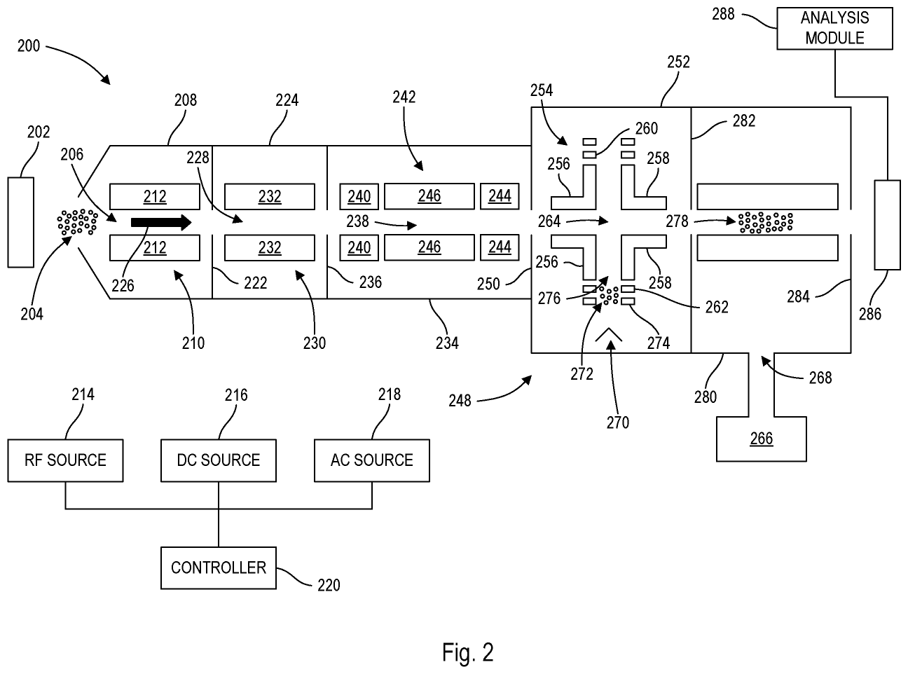

A mass spectrometric analysis method according to the present teachings can be implemented using a variety of mass spectrometers. By way of example, FIG. 2 schematically depicts such a mass spectrometer 200 that includes an electrospray ion source 202 that can receive a sample containing or suspected of containing one or more peptides of interest to ionize at least a portion of those peptides so as to produce a plurality of negatively charged peptide ions 204.

The charged peptide ions 204 pass through a QJet region 206 that is disposed within the chamber 208, which is the 1^{st} differential pumping stage. The QJet region 206 includes an ion guide 210, which in this embodiment includes four rods 212 that are arranged relative to one another in a quadrupole configuration.

The mass spectrometer 200 further includes an RF voltage source 214, a DC voltage source 216, and an AC voltage source 218 that are each under operation of a controller 220. The RF voltage source 214 can apply RF voltages to the rods 214 so as to generate an RF field, which in combination with gas dynamics can focus the charged peptide ions 204 into an ion beam for transmission to downstream components of the mass spectrometer.

The charged peptide ions 204 pass through the ion guide 212 and are further focused by an IQ0 lens 222 and enter a vacuum chamber 224, which is the 2^{nd} differential pumping stage. The charged peptide ions 204 continue in the direction of arrow 226 and travel through a Q0 region 228 that includes a second ion guide 230, which in this embodiment, includes four rods 232 that are arranged in a quadrupole configuration. The RF voltage source 214 is electrically connected to the rods 232 and supplies RF voltages to the rods 232 so as to generate an RF field for providing radial confinement of the ions 204 in proximity of the central axis of the rods 232.

The charged peptide ions 204 continue propagating in the direction of arrow 226 and enter a vacuum chamber 234 via an IQ1 ion lens 236. Once within the vacuum chamber 234, the charged peptide ions 204 pass through a Q1 region 238 that includes a stubby lens (Brubaker lens) 240, a mass filter 242, and a stubby lens (Brubaker lens) 244. The stubby lens 240 is positioned upstream from the mass filter 242 and the stubby lens 244 is positioned downstream from the mass filter 242. The mass filter 242 includes a plurality of rods 246 that are arranged in a multipole configuration. More specifically, in this embodiment, the mass filter 242 includes four rods 246 arranged in a quadrupole configuration. The stubby lens 240 focuses charged peptide ions 204 exiting the vacuum chamber 224 into the mass filter 242.

The RF voltage source 214 provides RF voltages to the rods 246 and the DC voltage source provides resolving DC voltages to the rods 246 of the mass filter 242. These voltages provide radial confinement of the ions 204 and further allow selecting ions 204 with a target m/z ratio or allows selecting ions 204 within a target range of m/z ratios to pass through the mass filter 242. After passing through the mass filter 242 the ions 204 are focused by the stubby lens 244 into a dissociation device 248 that is positioned downstream from the chamber 234. The ions 204 enter the dissociation device 248 via an IQ2 lens 250, which further focuses the charge peptide ions 204.

The dissociation device 250 includes a chamber 252 in which an ion trap 254 is disposed. The ion trap 254 is defined by first L-shaped electrodes 256 and second L-shaped electrodes 258 (also referred to as L-shaped rods 256 and 258, respectively) that are axially separated from one another, electrodes 260 and 262 (e.g., a lens electrode). At the center of the ion trap 254 within a gap formed by an axial separation of the L-shaped rods 256 and 258 is a reaction region 264 in which precursor ions can interact with an electron beam to undergo negative EAD, as discussed in more detail below.

In this embodiment, each of the first L-shaped electrodes 256 and the second L-shaped electrodes 258 include four electrodes (only two of which are shown in FIG. 1) that are arranged in a quadrupole configuration and are axially separated from one another to provide the ion reaction region 264 therebetween. In other embodiments, the first L-shaped electrodes 256 and the second L-shaped electrodes 258 may be arranged in other configurations (e.g., hexapole, octupole, dodecapole, etc.). The first L-shaped electrodes 256 and the second L-shaped electrodes 258 form an axial pathway (in the direction of arrow 226) through which the charged peptide ions 204 may pass through. Further, the arrangement of the first L-shaped electrodes 256 and the second L-shaped electrodes 258 also forms a transverse pathway that is perpendicular to the axial pathway. The ion trap 254 formed by the first L-shaped electrodes 256 and the second L-shaped electrodes 258 may be referred to as a “branched ion trap.”

The RF voltage source 214 and the DC voltage source 216 operating under control of the controller 220 supply voltages to the L-shaped electrodes 256 and 258 so as to trap the negatively charged peptide ions 204 within the ion trap 254. In embodiments, such as the present embodiment in which the first L-shaped electrodes 256 and second L-shaped electrodes 258 are supplied with RF voltages for providing radial confinement of the received ions, the ion trap 254 may be referred to as a “branched RF ion trap.”

The electrode 260 and an electrode 262 are positioned in proximity of the openings of the transverse pathway defined by the first L-shaped electrodes 256 and second L-shaped electrodes 258. The DC voltage source 216 can be used to apply a DC voltage to the electrodes 260 and 262 so as to maintain the electrodes 260 and 262 at an electric potential that would inhibit the negatively charged peptide ions 204 from leaking out of the ion trap 254 via the transverse pathway.

The mass spectrometer 200 includes a gas reservoir 266 that is in communication with the chamber 252. The gas reservoir 266 supplies a neutral buffer gas (e.g., neon, krypton, helium, nitrogen, argon, etc.) to the chamber 252 via an input port 268. In the ion trap, molecules of the neutral gas collide with the peptide ions 204 to cause collisional cooling thereof. By way of example, such collisions can reduce the kinetic and/or vibrational energy of the peptide ions

The mass spectrometer 200 also includes an electron source 270 (e.g., a filament) that generates a plurality of electrons 272 and a gate electrode 274. The gate electrode 274 and the pole electrode 262 are positioned between the electron source 270 and an inlet 276. In this embodiment, the mass spectrometer 200 can further include a magnet (not shown) that is configured to generate a magnetic field extending from the electron source 270 to the gate electrode 258 to confine the electrons 272.

The DC voltage source 216 can also apply DC voltages to the gate electrode 274 and the pole electrode 262 such that the gate electrode 274 is positively biased relative to the electron source 270. The bias of the electron source 270 is set in a range of about −20 to −50 volts relative to the ion trap 254. In these cases, the accelerated electrons can have a kinetic energy greater than at least 20 eV and specifically can have a kinetic energy in a range of about 20 eV to about 50 eV (e.g., 25 eV, 30 eV, 35 eV, 40 eV, 45 eV etc.) in the ion trap 254. In some embodiments, the controller 220 controls the temperature of the electron source 270 to increase or decrease a current associated with the emitted electrons 272. By way of example, the current generated by the electrons 276 may be in a range of about 10 to about 200 microamps.

The electrons 272 are introduced into the ion trap 254 as an electron beam via the inlet 276 of the transverse pathway. By way of example, the electron beam can have a diameter of about 1 mm. In the ion trap 254 the electrons 272 interact with the peptide ions 204 thereby causing the peptide ions 204 to fragment into fragment ions 278. Since the peptide ions 204 have a reduced vibrational state due to their collisions with molecules of the buffer gas, the fragment ions 278 include the fragile moieties.

In some embodiments, the electrons 272 create an electric potential within the ion trap 254. This electric potential may repel the negatively charged peptide ions 204 thereby reducing a number of collisions between negatively charged peptide ions 204 and electrons 272, and hence reduce the efficiency of the EDD of the negatively charged peptide ions 204. In these embodiments, the electrons 272 may ionize molecules of the buffer gas via electron impact ionization (EI), thereby generating a plurality of positively charged ions (e.g., N₂⁺, He⁺, Ne⁺, Kr⁺, etc.) within the reaction region 264 of the ion trap 254. The positive charge of the ionized buffer gas can neutralize the negative charge of the electrons 272 thereby providing a substantially electrically neutral plasma which can reduce and preferably eliminate the repulsive forces experienced by the negatively charged peptide ions 204. As a result, the provided neutral environment may lead to more efficient EDD as the neutral environment is more conducive to peptide ion 204 and electron 272 collision.

The pole electrodes 260 and 262 are negatively biased relative to the reaction region 264 of the ion trap 254. That is, the pole electrodes 260 and 262 are negatively biased relative to the first L-shaped electrodes 256 and second L-shaped electrodes 258. This negative bias of the pole electrodes prevents the peptide ions 204 from escaping the electron trap 254 via the inlet 276 while allowing the negatively charged electrons 272 to enter the ion trap 254.

Fragment ions of interest enter a downstream Q2 collision cell 280 via an aperture of an IQ2 lens 282. In the collision cell, fragment ions 278 collide with buffer gas molecules supplied by the gas reservoir 266. These collisions result in cooling of the fragment ions 278. The fragment ions 278 continue propagating in the direction of arrow 226 and exit the collision cell 280 via passage through an aperture of a lens 284.

The mass spectrometer 200 further includes a mass analyzer 286 (e.g., a time-of-flight (TOF) analyzer or another type of mass analyzer) positioned downstream from the collision cell 280 that receives the fragment ions 278 and provides mass spectral data associated with the fragment ions 278. An analysis module 288 receives the mass spectral data generated by the mass analyzer 286 and processes the data to generate a mass spectrum of the fragment ions 278 and correlates the mass spectrum of the fragment ions 278 with peptide ions 204 from which the fragment ions 278 were generated.

While the mass spectrometer 200 is described as including the collision cell 280 in other embodiments, the collision cell 280 may be omitted. In some such embodiments, the gas reservoir 266 is in communication with the chamber 252 such that the buffer gas is supplied directly into the chamber 252.

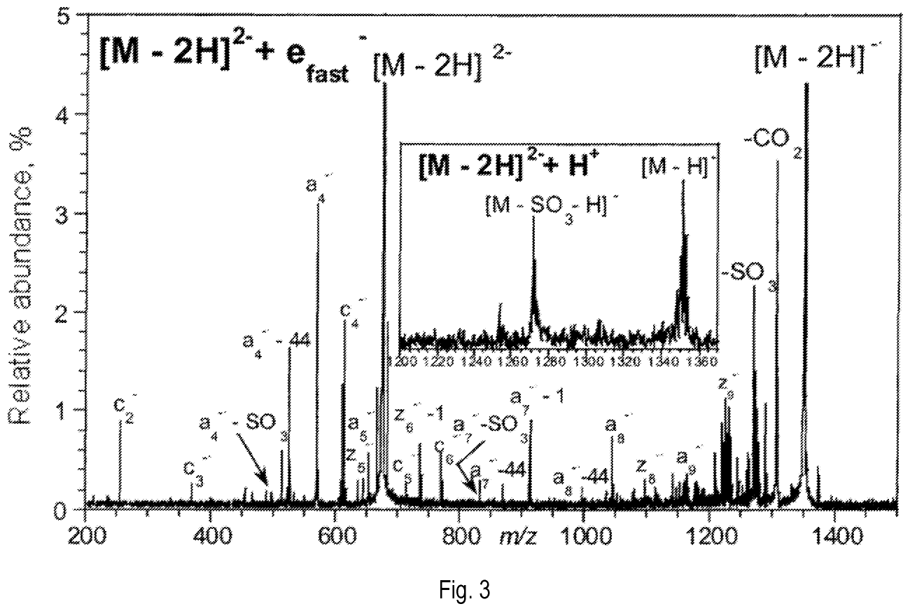

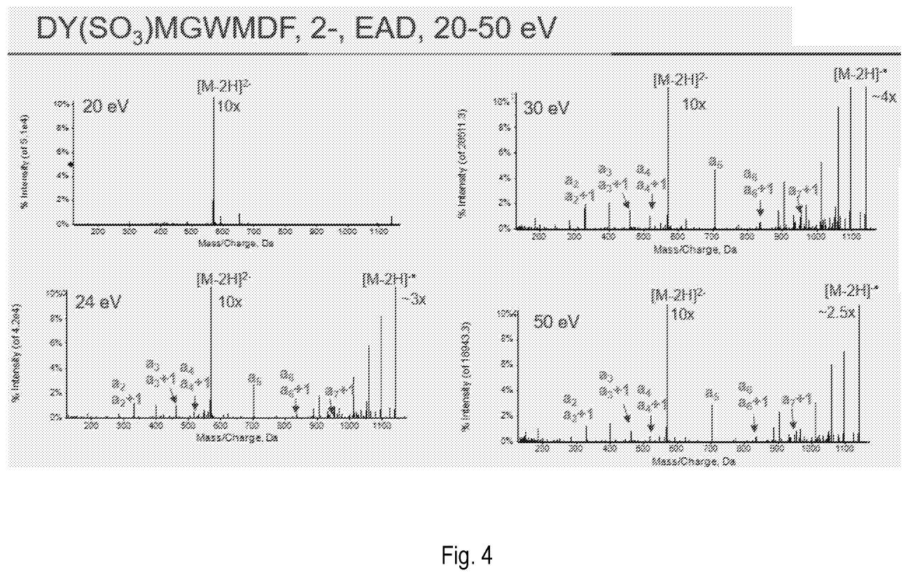

FIGS. 3 and 4 show MS/MS spectrums of sulfated peptide obtained via EDD. The mass spectrometer that generated the spectrum shown in FIG. 3 did not include a buffer gas within the ion trap (Observed in FT-ICR mass spectrometer. Chemical Physics Letters. Volume 342, Issues 3-4, 13 Jul. 2001, Pages 299-302.) whereas the mass spectrometer that generated the spectrum shown in FIG. 4 included a buffer gas within the ion trap (this disclosure). As shown in FIG. 3, the mass spectrometer used to generate the MS/MS spectrum (i.e., a mass spectrometer without a buffer gas within the ion trap) removes SO₃ from the precursor peptide ion whereas the mass spectrometer used to generate FIG. 4 (i.e., a mass spectrometer with a buffer gas within the ion trap). removed SO₃ from the precursor peptide ion to some extent, but fragments kept the sulfation.

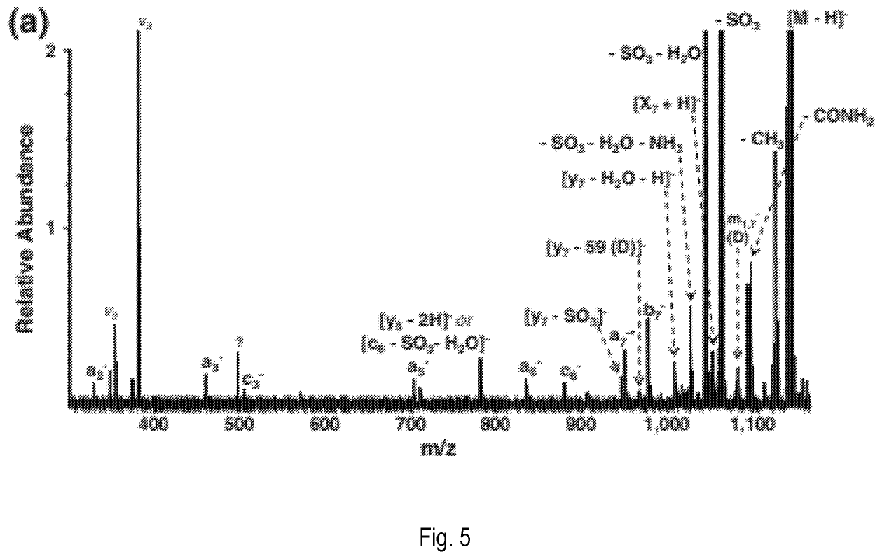

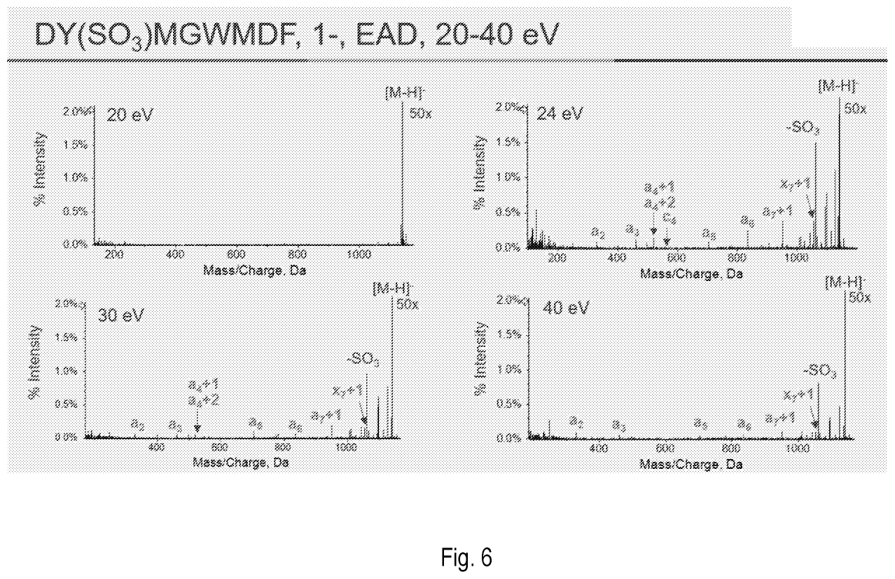

FIGS. 5 and 6 show MS/MS spectra of sulfated peptide obtained via EID. The mass spectrometer that generated the spectrum shown in FIG. 5 did not include a buffer gas within the ion trap (Observed in FT-ICR mass spectrometer. J. Am. Soc. Mass Spectrom. (2011) 22: page 2209-2221) whereas the mass spectrometer that generated the mass spectrum shown in FIG. 6 included a buffer gas within the ion trap (this disclosure). As shown in FIG. 5, the mass spectrometer used to generate the MS/MS spectrum (i.e., a mass spectrometer without a buffer gas within the ion trap) removed SO₃ from the precursor peptide ion whereas the mass spectrometer used to generate the mass spectrum shown in FIG. 6 (i.e., a mass spectrometer with a buffer gas within the ion trap) removed SO₃ from the precursor peptide ion to some extent, but fragments kept the sulfation.

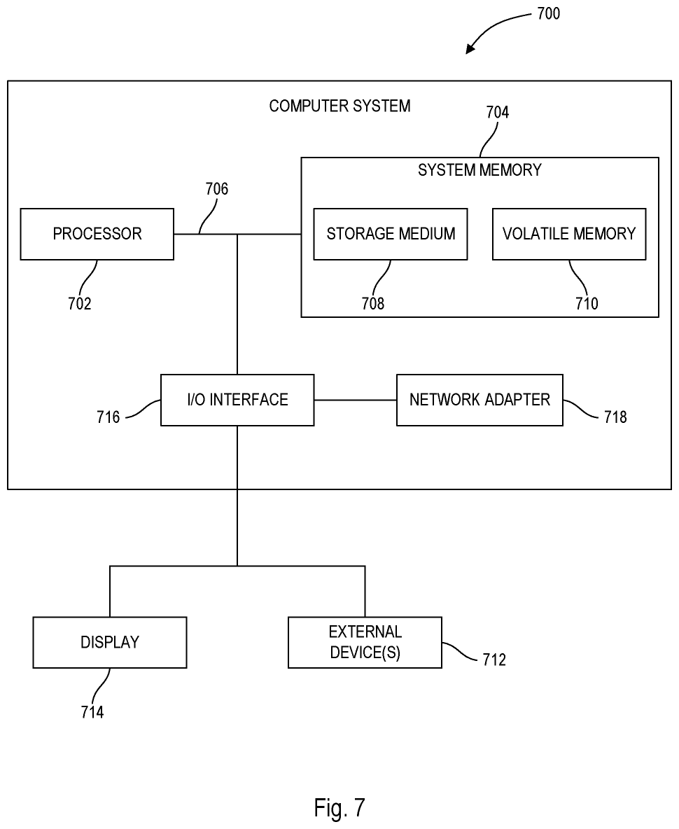

Referring now to FIG. 7 a computer system 700 is shown in accordance with an exemplary embodiment. In some embodiments, the computer system 700 serves as the controller 122.

As used herein a computer system (or device) is any system/device capable of receiving, processing, and/or sending data. Examples of computer systems include, but are not limited to personal computers, servers, hand-held computing devices, tablets, smart phones, multiprocessor-based systems, mainframe computer systems, and distributed cloud computing environments that include any of the above systems and the like.

As shown in FIG. 7, the computer system 700 includes one or more processors or processing units 702, a system memory 704, and a bus 706 that couples various components of the computer system 700 including the system memory 704 to the processor 706.

The system memory 704 includes a computer readable storage medium 708 and volatile memory 710 (e.g., Random Access Memory, cache, etc.). As used herein, a computer readable storage medium includes any media that is capable of storing computer readable program instructions and is accessible by a computer system. The computer readable storage medium 708 includes non-volatile and non-transitory storage media (e.g., flash memory, read only memory (ROM), hard disk drives, etc.). Computer readable program instructions as described herein include program modules (e.g., routines, programs, objects, components, logic, data structures, etc.) that are executable by a processor. Furthermore, computer readable program instructions, when executed by a processor, can direct a computer system (e.g., the computer system 700) to function in a particular manner such that a computer readable storage medium (e.g., the computer readable storage medium 708) comprises an article of manufacture. Specifically, when the computer readable program instructions stored in the computer readable storage medium 708 are executed by the processor 702, they create means for implementing various functions disclosed herein.

The bus 706 may be one or more of any type of bus structure capable of transmitting data between components of the computer system 700 (e.g., a memory bus, a memory controller, a peripheral bus, an accelerated graphics port, etc.).

In some embodiments, as depicted in FIG. 7, the computer system 700 may include one or more external devices 712 and a display 714. As used herein, an external device includes any device that allows a user to interact with a computer system (e.g., mouse, keyboard, touch screen, etc.). An external device 712 and the display 714 are in 716 with the processor 702 and the system memory 704 via an Input/Output (I/O) interface 716.

The display 714 may show a graphical user interface (GUI) that may include a plurality of selectable icons and/or editable fields. A user may use an external device 712 (e.g., a mouse) to select one or more icons and/or edit one or more editable fields. Selecting an icon and/or editing a field may cause the processor 702 to execute computer readable program instructions stored in the computer readable storage medium 704. In one example, a user may use an external device 712 to interact with the computer system 700 and cause the processor 702 to execute computer readable program instructions relating to various functions disclosed herein.

The computer system 700 may further include a network adapter 718 which allows the computer system 700 to communicate with one or more other computer systems/devices via one or more networks (e.g., a local area network (LAN), a wide area network (WAN), a public network (the Internet), etc.).

While various embodiments have been illustrated and described in detail in the drawings and foregoing description, such illustration and description are to be considered illustrative or exemplary and not restrictive; embodiments of the present disclosure are not limited to the disclosed embodiments. Other variations to the disclosed embodiments can be understood and effected by those skilled in the art in practicing embodiments of the present disclosure, from a study of the drawings, the disclosure, and the appended claims.

In the claims, the word “comprising” does not exclude other elements or steps, and the indefinite article “a” or “an” does not exclude a plurality. A single processor or other processing unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

1. A method for mass spectrometric analysis of a peptide having at least one fragile moiety, comprising:
using electrospray ionization to generate a negatively charged ion of said peptide,
trapping and cooling the negatively charged peptide ion in a radiofrequency (RF) ion trap containing a cooling buffer gas, and
exposing said cooled, trapped peptide ion to an electron beam so as to cause negative electron activated dissociation (negative EAD) of the negatively charged peptide ion to generate a plurality of fragment ions.

2. The method of claim 1, wherein said negative EAD comprises any of electron detachment dissociation (EDD) and electron impact dissociation (negative EID).

3. The method of claim 1, wherein said electron beam has a kinetic energy in a range of about 20 eV to about 50 eV.

4. The method of claim 1, wherein said RF ion trap comprises a linear RF ion trap.

5. The method of claim 4, wherein said linear RF ion trap comprises a plurality of rods arranged in a multipole configuration.

6. The method of claim 5, wherein said multipole configuration comprises any of a quadrupole configuration, a hexapole configuration, an octupole configuration, and a dodecapole configuration.

7. The method of claim 1, wherein said RF ion trap comprises a branched RF ion trap.

8. The method of claim 1, wherein said cooling buffer gas comprises any of nitrogen, helium, neon, argon, and xenon.

9. The method of claim 1, further comprising acquiring a mass spectrum of the fragment ions.

10. A mass spectrometer, comprising:
an ion source configured to ionize a plurality of peptides that include a fragile moiety thereby generating a plurality of peptide ions that include a fragile moiety; and
a chamber including:
an ion trap that includes a buffer gas and is configured to trap the peptide ions, and
an electron source configured to generate a plurality of electrons in the form of an electron beam and configured to introduce the electron beam into the ion trap,
wherein molecules of the buffer gas cool the peptide ions within the ion trap, and
wherein the electron beam interacts with at least a portion of the peptide ions to cause negative EAD thereby generating a plurality of fragment ions with a fragile moiety.

11. The mass spectrometer of claim 10, wherein peptide ion is a negative ion.

12. The mass spectrometer of claim 10, wherein negative EAD is one of EDD or EID.

13. The mass spectrometer of claim 10, wherein the electron beam has a kinetic energy in a range of about 20 eV to about 50 eV.

14. The mass spectrometer of claim 10, wherein the ion trap is a linear RF ion trap.

15. The mass spectrometer of claim 14, wherein the linear RF ion trap comprises a plurality of rods arranged in a multipole configuration.

16. The mass spectrometer of claim 15, wherein the multipole configuration comprises any of a quadrupole configuration, a hexapole configuration, an octupole configuration, and a dodecapole configuration.

17. The mass spectrometer of claim 10, wherein said ion trap comprises a branched RF ion trap.

18. The mass spectrometer of claim 10, wherein said buffer gas comprises any of nitrogen, helium, neon, argon, and xenon.

19. The mass spectrometer of claim 10, further comprising:
a mass analyzer configured to receive the fragment ions and provide mass spectral data indicative of the fragment ions; and
a mass analysis module configured to process the mass spectral data to generate a mass spectrum of the fragment ions.

20. A chamber for use in a mass spectrometer, comprising:
an ion trap including:
a plurality of negatively charged peptide ions that include a fragile moiety, and
a buffer gas configured to reduce a vibrational state of the peptide ions; and
an electron source that is external from the ion trap and is configured to introduce an electron beam into the ion trap, wherein the electron beam interacts with the peptide ions to generate a plurality of fragment ions with a fragile moiety.
